(19) United States
(12) Patent Application Publication
(10) Pub. No.: US 2007/0219411 A1
(43) Pub. Date: Sep. 20, 2007

(54) Overtube and endoscopic treatment system

(75) Inventors: Takumi Dejima, Tokyo (JP); Manabu Miyamoto, Tokyo (JP); Kiyotaka Matsuno, Sagamihara-shi (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 11/649,099

(22) Filed: Jan. 3, 2007

Related U.S. Application Data
(63) Continuation-in-part of application No. 11/331,938, filed on Jan. 13, 2006.

(51) Int. Cl. A61B 1/00

(52) U.S. Cl. 600/141

(57) ABSTRACT

An overtube includes an insertion part that is inserted into a subject and has a lumen through which a device inserting part of a device whose distal end bends freely for performing a medical procedure inside a body is removably inserted and a bending part that bends the distal end side of the lumen, in which the bending part is provided with a bending tube having a plurality of joint rings that are connected via connecting shafts along the lumen to freely turn and a braided tube that is disposed on the inner side of the bending tube and forms the periphery of the lumen.

Priority is claimed on U.S. patent application Ser. No. 11/331,938, filed Jan. 13, 2006, the content of which is incorporated herein by reference.

### BACKGROUND OF THE INVENTION

1. Field of the Invention

This invention relates to an overtube and an endoscopic treatment system.

2. Description of Related Art

Laparoscopic operations are known in which, in performing a medical procedure of observing, treating, etc. an organ of the human body, instead of incising the abdominal wall widely, a plurality of orifices are opened in the abdominal wall and procedures are performed upon inserting a laparoscope, forceps, and other treatment instruments into the orifices. Such procedure provides the benefit of lessening the burden placed on the patient because only small orifices need to be opened in the abdominal wall.

In recent years, methods of performing procedures upon inserting a flexible endoscope via the mouth, nose, anus, or other natural orifice of the patient have been proposed as methods of further reducing the burden on the patient. An example of such procedures is disclosed in U.S. Pat. No. 5,458,131.

With this method, a flexible endoscope is inserted from the mouth of a patient, an opening is formed in the stomach wall, and a distal end part of the endoscope is fed into the abdominal cavity from the opening. Then while using the endoscope as a device for observing the interior of the abdominal cavity, desired procedures are performed inside the abdominal cavity using a treatment instrument inserted through the endoscope or a treatment instrument inserted from another opening.

An object of this invention is to provide an overtube that enhances the ability to insert an endoscope and an endoscopic treatment system.

### SUMMARY OF THE INVENTION

An overtube according to a first aspect of this invention includes an insertion part inserted into a subject having a lumen through which a device inserting part of a device whose distal end bends freely for performing a medical procedure inside a body is removably inserted and a bending part that bends the distal end side of the lumen, in which the bending part is provided with a bending tube having a plurality of joint rings that are connected via connecting shafts along the lumen to freely turn and a braided tube that is disposed on the inner side of the bending tube and forms the periphery of the lumen.

Also, the endoscopic treatment system according to a second aspect of this invention includes: an overtube according to the first aspect of this invention; a device inserting part that is inserted in the insertion part of the overtube in which is provided a treatment instrument insertion channel whose distal end is opened; and a puncture needle that is inserted in the treatment instrument insertion channel, with the distal end thereof splitting apart to be wider than the inner diameter of the treatment instrument insertion channel.

### BRIEF DESCRIPTION OF THE DRAWINGS

FIG. 1 is a schematic view of an entirety of an overtube according to a first embodiment.

FIG. 2 is a view of the principal portions of the overtube according to the first embodiment.

FIG. 3 is a view of the principal portions of the overtube according to the first embodiment.

FIG. 4 is a sectional view taken along line A-A of FIG. 2.

FIG. 5 is a sectional view taken along line B-B of FIG. 4.

FIG. 6 is a view showing the constitution of the inner braid of the overtube according to the first embodiment.

FIG. 7 is a view showing the principal portions of the puncture needle used with the endoscope system according to the first embodiment.

FIG. 8 is an overall view of the double T-bars used with the endoscope system according to the first embodiment.

FIG. 9 is a sectional view of a state in which the double T-bars are fitted into a puncture needle according to the first embodiment.

FIG. 10 is a partial sectional view showing the manipulating part of the puncture needle according to the first embodiment.

FIG. 11 is an overall schematic view of an endoscope as an example of a device used with the endoscope system according to the first embodiment.

FIG. 12 is a flowchart of a medical procedure according to the first embodiment.

FIG. 13 is a view for describing a state of inserting the endoscope into the overtube in the medical procedure according to the first embodiment.

FIG. 14 is a view for describing a state of grasping an incision target site with grasping forceps in the medical procedure according to the first embodiment.

FIG. 15 is a view for describing a state of insufflating by feeding air from an injection needle in the medical procedure according to the first embodiment.

FIG. 16 is a view for describing a state of the puncture needle accommodated in the treatment instrument insertion channel in the medical procedure according to the first embodiment.

FIG. 17 is a view for describing a state of protruding the puncture needle from the treatment instrument insertion channel in the medical procedure according to the first embodiment.

FIG. 18 is a view for describing a state of the double T-bars being retained in the puncture needle in the medical procedure according to the first embodiment.

FIG. 19 is a view for describing a state of the puncture needle piercing the incision target site while retaining the double T-bars in the medical procedure according to the first embodiment.

FIG. 20 is a view for describing a state releasing the anchors of the double T-bars from the puncture needle and incising the incision target site with a high-frequency knife in the medical procedure according to the first embodiment.

FIG. 21 is a view for describing the state of incising the incision target site while grasping it with the grasping forceps.

FIG. 22 is a view for describing the state of having incised the incision target site in the case of FIG. 20.

FIG. 23 is a view for describing the state of having incised the incision target site in the case of FIG. 21.

FIG. 24 is a view for describing the state of the endoscope being inserted in the abdominal cavity in the medical procedure according to the first embodiment.

FIG. 25 is a view for describing the state of pulling and tensioning the suture of the placed double T-bars in the medical procedure according to the first embodiment.

FIG. 26 is a view for describing the action in FIG. 25.

FIG. 27 is a view for describing the state of the stomach wall being bound with the double T-bars in the medical procedure according to the first embodiment.

FIG. 28 is a view of the principal portions of the overtube according to the second embodiment.

FIG. 29 is a perspective sectional view of the overtube according to the second embodiment.

FIG. 30 is a view showing the action of the principal portions of the overtube according to the second embodiment.

FIG. 31 is a perspective sectional view showing the action of the overtube according to the second embodiment.

FIG. 32 is a view showing the constitution according to a modification example of the overtube according to the first embodiment.

FIG. 33 is a view showing the principal portions of a modification example of the overtube according to the second embodiment.

FIG. 34 is a view showing the action of the principal portions of the overtube according to the second embodiment.

### DETAILED DESCRIPTION OF THE INVENTION

Embodiments according to the present invention will now be described in detail below. In the following description, components that are the same shall be provided with the same numeric symbol and redundant description shall be omitted.

### First Embodiment

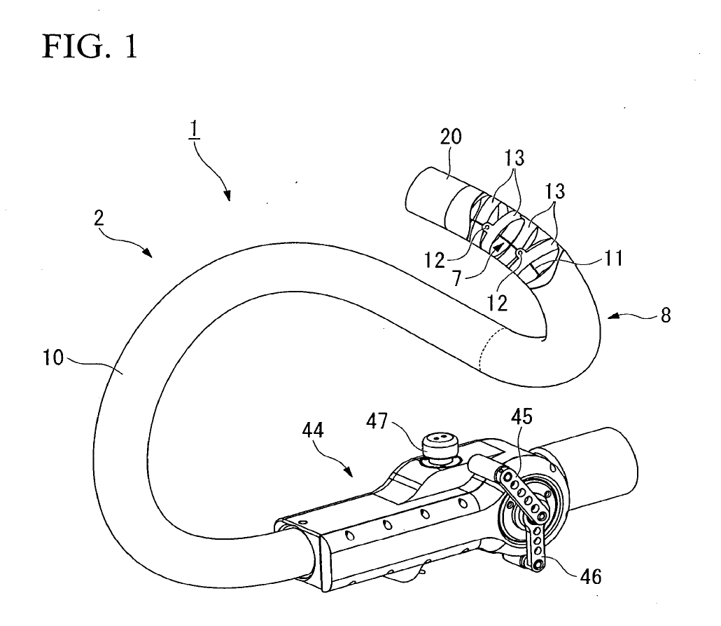

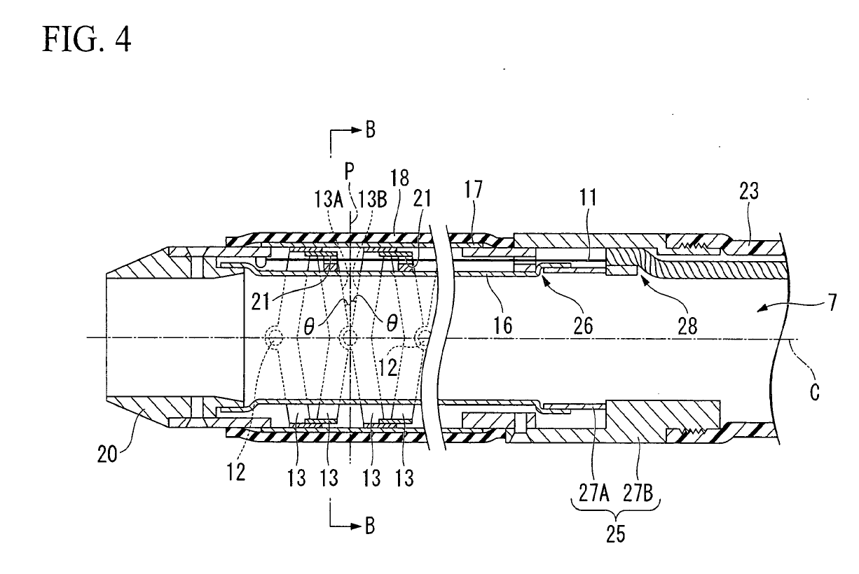

An endoscopic treatment system 1 according to the present embodiment, as shown in FIG. 1 to FIG. 11, includes: an overtube 2; an endoscope (device) 3 that is inserted in the overtube 2 for carrying out a medical procedure inside a body; and a puncture needle 6 that is inserted through treatment instrument insertion channels 58 and 60 described below that are provided in an endoscope inserting part (device inserting part) 5 of the endoscope 3 whose distal end bends freely, and the distal end of the puncture needle splits apart to be wider than the inner diameter of the treatment instrument insertion channels 58 and 60.

An overtube 2 is used as a guide tube for inserting the endoscope 3 into a body. The overtube 2 includes: an insertion part 10 that is inserted into a stomach or other hollow organ or abdominal cavity, etc., of a patient (subject) and has a lumen 7 through which the endoscope inserting part 5 is removably inserted and a bending part 8 that bends the distal end side of the lumen 7; and a bending wire 11 for performing a bending operation of the bending part 8.

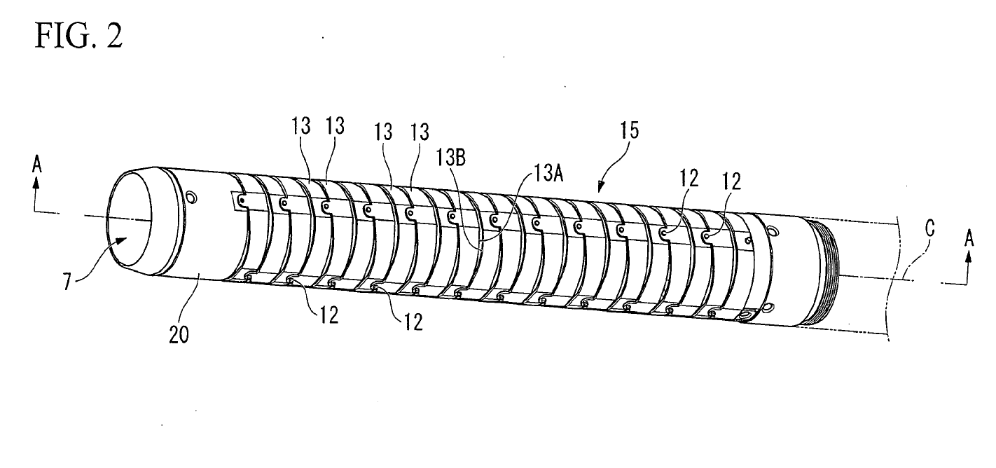

The bending part 8 is disposed on the distal end side of the insertion part 10 and, as shown in FIG. 2 to FIG. 4, includes a bending tube 15 that has of a plurality of ring-shaped joint rings 13 that are mutually connected via connecting shafts 12 along the lumen 7 to freely turn; a tubular inner braid (braided tube) 16 that is disposed on the inner side of the bending tube 15 and forms the periphery of the lumen 7; a tubular outer braid 17 that covers the periphery of the bending tube 15; and a resin outer skin 18 that constitutes the outermost layer of the bending part 8. A tubular distal end part 20 to which the distal end of the bending wire 11 is connected is connected to the distal end of the bending part 8.

Each joint ring 13 has a proximal-end side first surface 13A and a distal-end side second surface 13B. When the bending tube 15 bends, with respect to a virtual plane P that includes the connecting shafts 12 and is perpendicular to a central axis line C of the lumen 7, the first surface 13A and the second surface 13B incline respectively at a predetermined angle θ in the direction of the central axis C. When the bending tube 15 bends, the first surface 13A of the joint ring 13 and the second surface 13B of the adjacent joint ring 13 abut. Here, since the angle θ is an angle smaller than that of ordinary joint rings not shown that the endoscope inserting part 5 has, the gap between the joint rings 13 is narrower than usual. Also, in order to ensure the bending range of the bending tube 15, the number of joint rings 13 is more than normal.

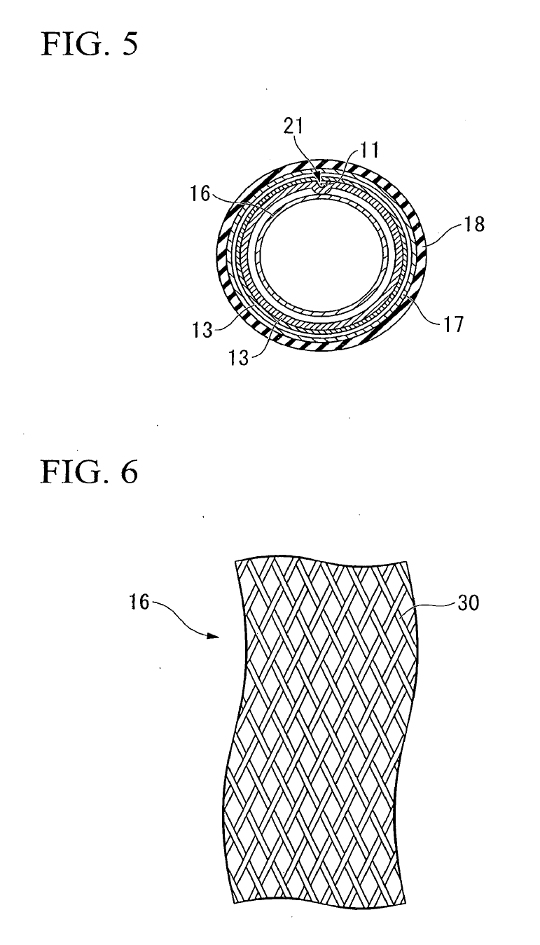

On the joint ring 13, a pass-through part 21 is provided for the bending wire 11 to pass through the bending tube 15 along the central axis C. The pass-through part 21, as shown in FIG. 5, is formed with a portion of the joint ring 13 being bent inward in the radial direction. For that reason, a portion of the inner braid 16 is mounted in a deformed state by being pressed inward in the radial direction by the pass-through part 21. The pass-through part 21 is provided at only one location. That is, one bending wire 11 only is disposed in the pass-through part 21. The bending tube 15 is constituted to bend only in the direction in which the side on which the bending wire 11 is inserted serves as the inner side in the radial direction. The bending wire 11 is disposed to freely advance and retract in a coil tube 22 further to the proximal end side than the bending part 8.

The insertion part 10 further to the proximal end side than the bending part 8 is covered by a resin layer 23. The distal end of the resin layer 23 and the bending part 8 are connected via a connecting part 25. The connecting part 25 is provided with an inner tube part 27A, on which the proximal end of the outer braid 17 is externally fitted, and an outer tube part 27B, on which the proximal end of the outer skin 18 is bonded and the distal end of the resin layer 23 is screw fitted. The inner tube part 27A is provided with a first slit 26 that sandwiches the proximal end of the inner braid 16, and the outer tube part 27B is provided with a second slit 28 that sandwiches the distal end of the coil tube 22.

The inner braid 16 and the outer braid 17, as shown in FIG. 6, are formed by braiding one thin metallic wire 30 so as to intersect with the central axis C. For that reason, the gaps that are formed between the joint rings 13 are blocked by the inner braid 16.

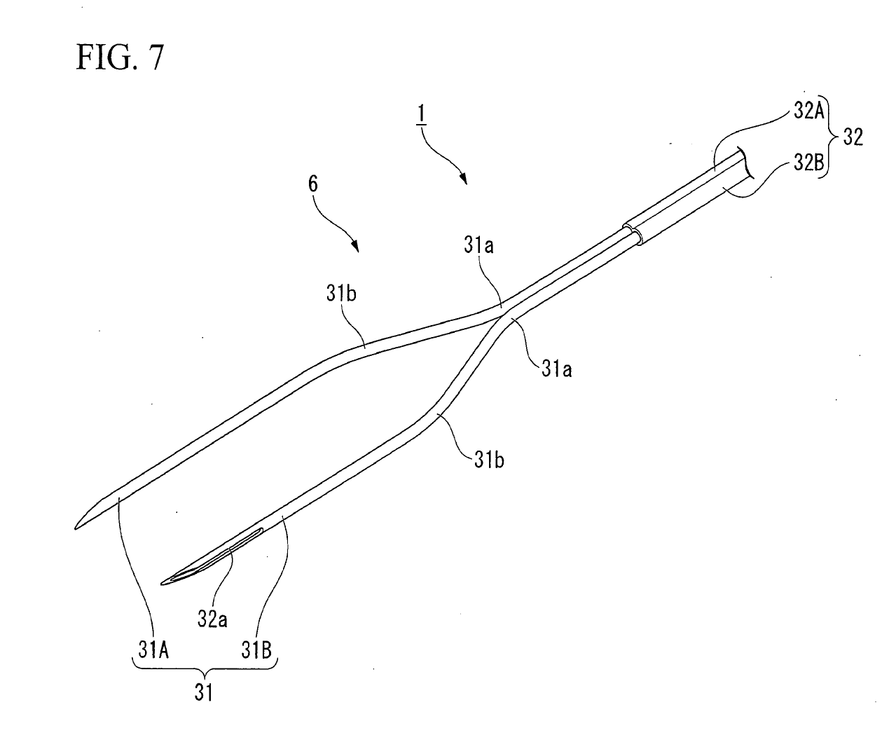

As shown in FIG. 7, the puncture needle 6 includes: a needle part 31 that has a metal first needle part 31A and a second metal needle part 31B that are hollow and spaced apart; and a sheath 32 that has a first sheath 32A and a second sheath 32B that respectively accommodate the first needle part 2A and the second needle part 2B to freely protrude and retreat.

The first needle part 31A and the second needle part 31B are each provided with a bend part 31a that separates a distal end side of the first needle part 31A and the second needle part 31B to be further apart than the gap between a proximal end side thereof. Further to the distal end side than the bend part 31a of the first needle part 31A and the second needle part 31B is also provided an alignment part 31b that disposes the distal end sides of the first needle part 31A and the second needle part 31B to be mutually parallel. A slit 32a through which a suture 33C described below passes is formed at the distal end of the first needle part 31A and the second needle part 31B. The bend part 31a and the alignment part 31b resiliently deform to be accommodated in the sheaths 32A and 32B when accommodating the needle parts 31A and 31B in the sheaths 32A and 32B. At least the distal end sides of the sheaths 32A and 32B are integrated so as not to come apart.

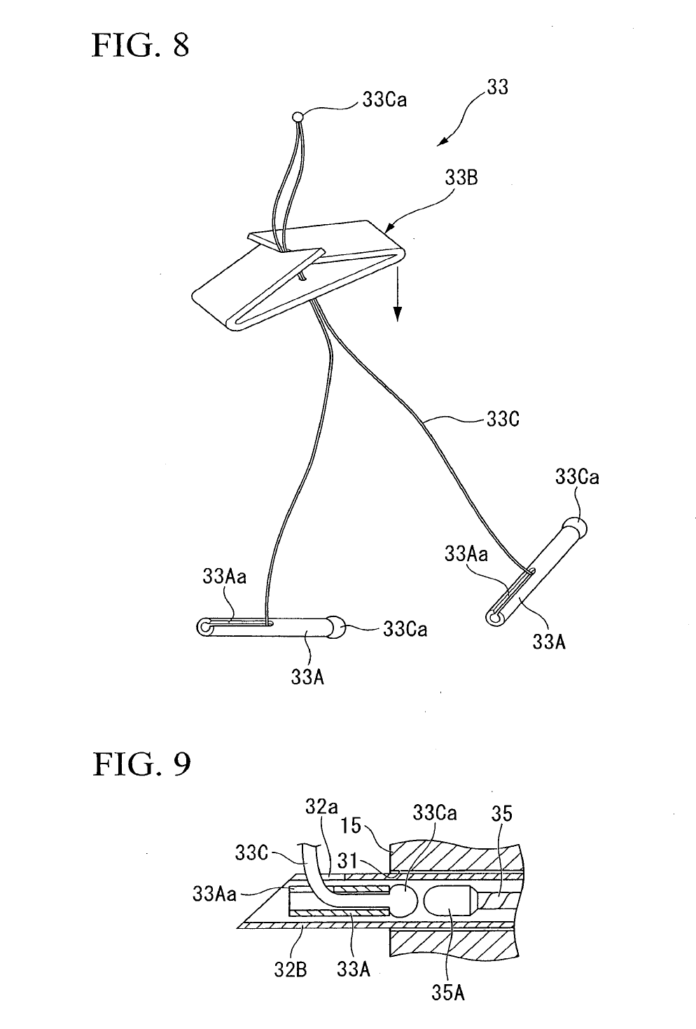

Two anchors 33A of double T-bars 33, shown in FIG. 8, are respectively held inside the respective needle parts 31A and 31B. The double T-bars 33 have two sutures 33C, one end side of each of which is passed through a substantially triangular stopper 33B. At one end, the sutures 33C are bound together to form a large diameter part 33Ca. Each of the other ends of the sutures 33C is fixed to the anchors 33A. Each anchor 33A has a cylindrical shape with a slit formed at an end, and the suture 33C is inserted in the longitudinal direction of the interior of the anchor 33A through the slit. The large diameter part 33Ca that has greater diameter than that of the anchor 33A is formed at the other end of the suture 33C. The stopper 33B has a hole, through which the sutures 33C are passed, at a center in the longitudinal direction of an elongated, thin plate member. The respective ends in the longitudinal direction of the stopper 33B are folded obliquely and sandwich the sutures 33C. The respective ends in the longitudinal direction of the stopper 33B are cut to notches of triangular shape. With the stopper 33B, the respective ends are folded back obliquely so that the notches intersect and thereby sandwich the sutures 33C. The sutures 33C thus do not fall off from between the ends. When the large diameter part 33Ca of the sutures 33C is pulled in a direction away from the stopper 33B, the respective end parts of the stopper 33B spread apart slightly. The stopper 33B thus allows movement of the sutures 33C in this direction. Meanwhile, when a large diameter part 33Ca at the anchor 33A side of a suture 33C is pulled, a tendency for the suture 33C to move in the direction indicated by the arrow in FIG. 8 arises. However, since the respective ends of the stopper 33B close and grasp the sutures 33C in this process, the suture 33C does not move. A pusher 35 is movably disposed in advancing and retracting directions in the interior of the respective needle parts 31A and 32B. A rigid, pushing member 35A is disposed at a distal end of the pusher 35.

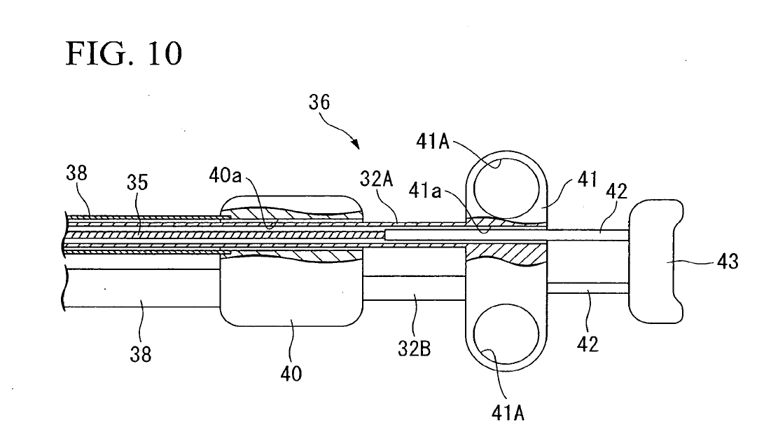

As shown in FIG. 10, the puncture needle 6 is provided with a needle manipulating part 36 that simultaneously protrudes and retracts the first needle part 31A with respect to the distal end of the first sheath 32A and the second needle part 31B with respect to the distal end of the second sheath 32B. The needle manipulating part 36 includes a sheath holding part 40 connected to the proximal ends of the first sheath 32A and the second sheath 32B; a needle manipulating handle 41 connected to proximal ends of the two needle parts 31 A and 31 B that have been passed in a manner enabling advancing and retracting through through-holes 40a formed in the sheath holding part 40; and a pusher connection part 43 that connects end portions of rod-like, rigid parts 42, which are passed in a manner enabling advancing and retracting through through-holes 41 a formed in the needle manipulating handle 41 and are connected to proximal ends of the two pushers 35, to each other. The needle manipulating handle 41 is provided with finger rings 41A. Each of the needle manipulating handle 41 and the pusher connection part 43 may be divided into two parts so as to enable the two needle parts 31A and 31B and the two pushers 35 to be manipulated independently of each other.

As shown in FIG. 1, a proximal handle 44 having a larger diameter than the insertion part 10 is disposed at the proximal end of the insertion part 10 of the overtube 2. The proximal handle 44 includes a bending lever 45, a bending lock lever 46, and an endoscope lock button 47. The bending lever 45 is connected to the proximal end side of the bending wire 11 for performing bending manipulation of the bending part 8. The bending lock lever 46 is used for fixing the position of the bending lever 45 at an arbitrary position. The endoscope lock button 47 is used for fixing the endoscope 3 with respect to the lumen 7 upon insertion of the endoscope 3 through the lumen 7.

Regarding the endoscope lock button 47, when the endoscope 3 must be fixed to the insertion part 10 upon being inserted through the interior, pressing the endoscope lock button 47 inward in the radial direction presses and fixes the endoscope inserting part 5 in a relative manner by a frictional force. The endoscope lock button 47 may be arranged so as to oppositely release the frictional force when pressed.

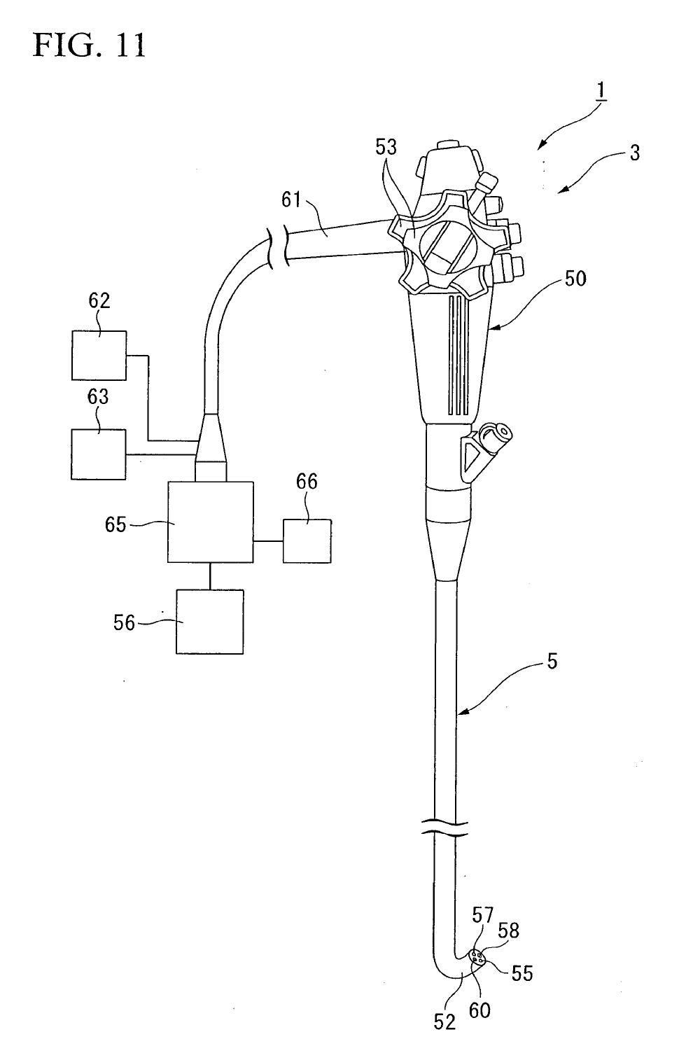

The endoscope 3 to be inserted in the overtube 2 is a flexible endoscope 13 as shown for example in FIG. 11. This endoscope 3 has an endoscope inserting part 5, which is elongated and has flexibility to be inserted into a patient's body, that extends outward from the endoscope manipulating part 50 manipulated by an operator. An endoscope distal end part 52 of the endoscope inserting part 5 can be bent by manipulating an angle knob 53 disposed at the endoscope manipulating part 50. At the endoscope distal end part 52 are disposed an objective lens 55, a distal end face of an optical fiber 57 that guides light from a light source device 56 disposed outside the body, and distal end openings of treatment instrument insertion channels 58 and 60. The treatment instrument insertion channels 58 and 60 are ducts for inserting and removing a treatment instrument. Moreover, the treatment instrument insertion channel 58 is connected via a universal cable 61 to an air/water feeding device 62 or a suction device 63 disposed outside the body. The treatment instrument insertion channel 60 is disposed at a position of six o'clock to eight o'clock of the endoscope inserting part 5.

An observation image input into the objective lens 55 is displayed on a monitor 66 via a control unit 65.

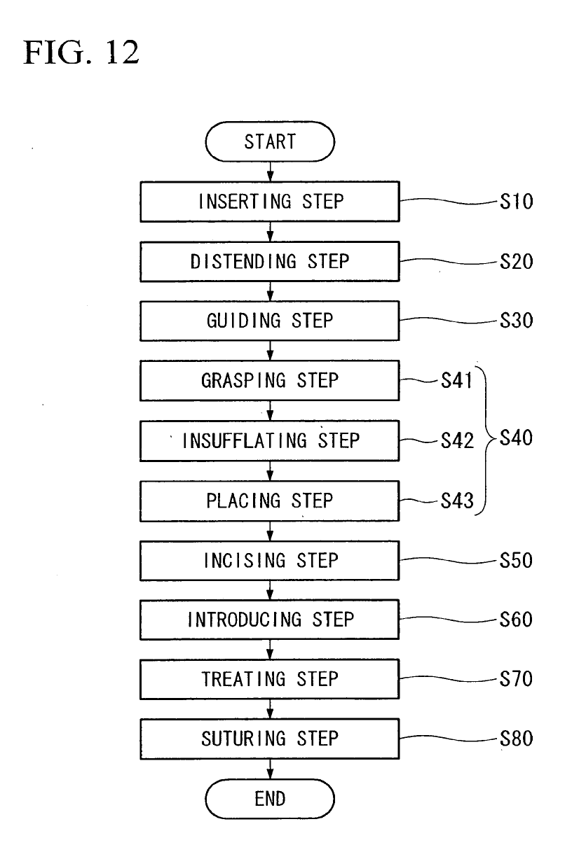

Actions of the present embodiment shall now be described in line with a medical procedure performed via a natural orifice as shown by the flow chart of FIG. 12. In the following description, it shall be deemed that an incision target site is located on an anterior wall of a stomach, and a surgical procedure of inserting the endoscope 3 into the stomach from a mouth of a patient and performing treatment upon forming an opening in the stomach wall and inserting the endoscope inserting part 5 into an abdominal cavity shall be described. Also, though in the embodiment described below, the endoscope 3 is introduced into the body from the mouth of the patient and made to approach the abdominal cavity upon forming the opening in the anterior wall of the stomach, the natural orifice from which the endoscope 3 is introduced is not restricted to the mouth and may be another natural orifice, such as the anus, nose, etc. Furthermore, though the forming of the opening in the anterior wall of the stomach is desirable, this invention is not restricted thereto, and an opening may be formed on the wall of other hollow organ (hollow organ) into which a device is introduced via a natural orifice, such as another area of the stomach, the esophagus, small intestine, or large intestine.

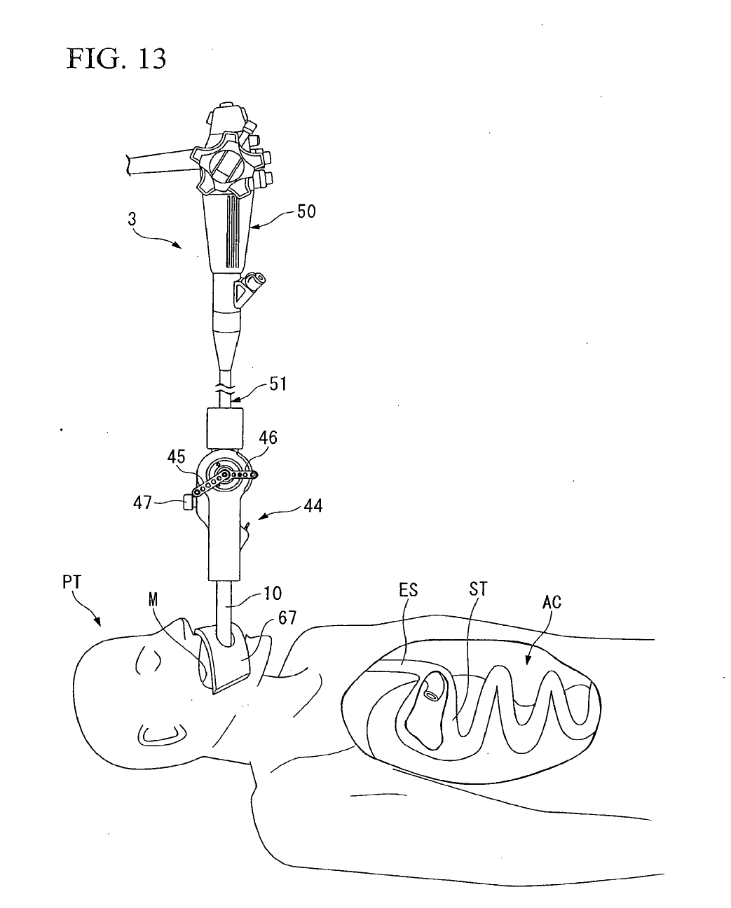

First, as shown in FIG. 13, with the patient PT being made to lie in a supine position, an inserting step (S10) of inserting the endoscope inserting part 5 through the lumen 7 in the insertion part 10 of the overtube 2 and inserting the insertion part 10 of the overtube 2 and the endoscope inserting part 5 into the stomach ST from the mouth M of the patient PT while observing the interior of the body cavity by means of an endoscopic image is performed. A mouthpiece 67 is fitted onto the mouth of the patient PT and the overtube 2 and the endoscope 3 are inserted, with the endoscope inserting part 5 being inserted through the interior of the lumen 7, into the esophagus ES from the mouthpiece 67.

Here, the inner braid 16 forms the inner periphery of the lumen 7. For this reason, when inserting the endoscope inserting part 5 into the lumen 7, even when the distal end thereof passes the bending part 8, the distal end of the endoscope inserting part 5 does not enter the gaps between the joint rings 13. At this time, since only one pass-through part 21 is provided in the joint rings 13, there is only one location of encroaching the inner diameter of the inner braid 16. Accordingly, a sufficiently large diameter of the lumen 7 is ensured, and so the endoscope inserting part 5 smoothly moves in the lumen 7 with the inner braid 16 serving as a guide.

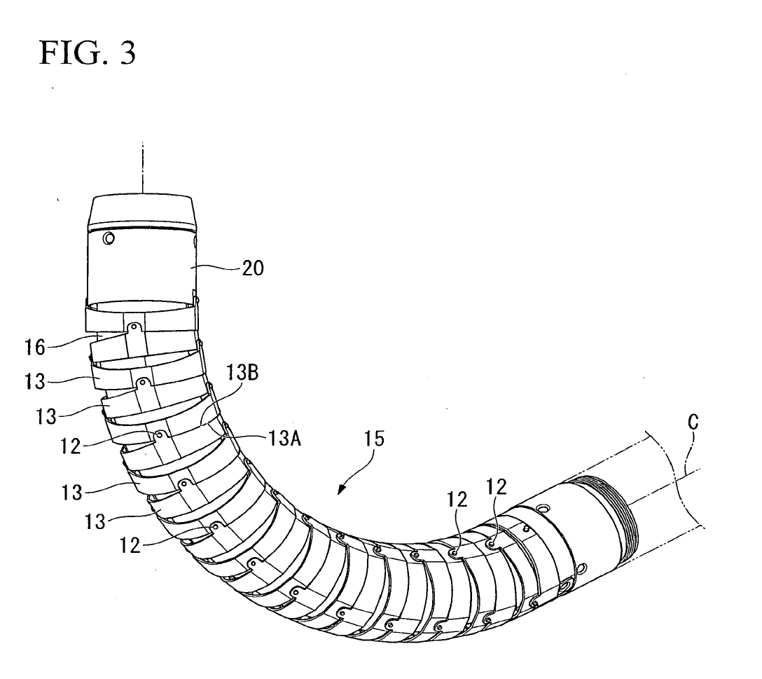

When bending the bending part 8, the bending wire 11 is pulled toward the proximal side. At this time, the joint rings 13 turn from the distal end side at a predetermined angle about the connecting shafts 12. Thereby, as shown in FIG. 3, bending occurs until the first surface 13A of the joint ring 13 makes contact with the second surface 13B of the opposing joint ring 13. When all the joint rings 13 similarly turn about the connecting shafts 12, the bending part 8 is formed having a prescribed curve.

On the other hand, to extend the bending part 8 so as to make it straight, the bending wire 11 is loosened. At this time, due to the resiliency of the endoscope inserting part 5, torque is added in the direction in which the first surface 13A and the second surface 13B of the joint rings 13 separate. The joint rings 13 thereby turn about the connecting shafts 12 in the opposite direction to the direction during bending, so that, as shown in FIG. 2, the bent bending tube 15 is straightened. Accordingly, the bending part 8 itself also becomes straightened.

Next, in a distending step (S20), air is supplied from the air/water feeding device 62 via the treatment instrument insertion channel 58 of the endoscope inserting part 5 to inflate the stomach ST.

A guiding step (S30) of guiding the insertion part 10 of the overtube 2 to the incision target site T while checking the incision target site T using the endoscope 3, which is also an observation device, is then performed. First, after inserting the endoscope inserting part 5 of the endoscope 3 into the stomach ST, the angle knob 53 is manipulated to bring the distal end of the endoscope inserting part 5 close to the incision target site T while observing the interior of the stomach ST via the objective lens 55, disposed at the endoscope inserting part 5. Then with the incision target site T being specified, the endoscope inserting part 5 is used as a guide to push the insertion part 10 of the overtube 2 and bring the distal end part 20 of the overtube 2 close to the incision target site T.

A needle moving step (S40) of making the needle part 31 of the puncture needle 6 puncture the stomach wall SW and placing the double T-bars 33 is then performed.

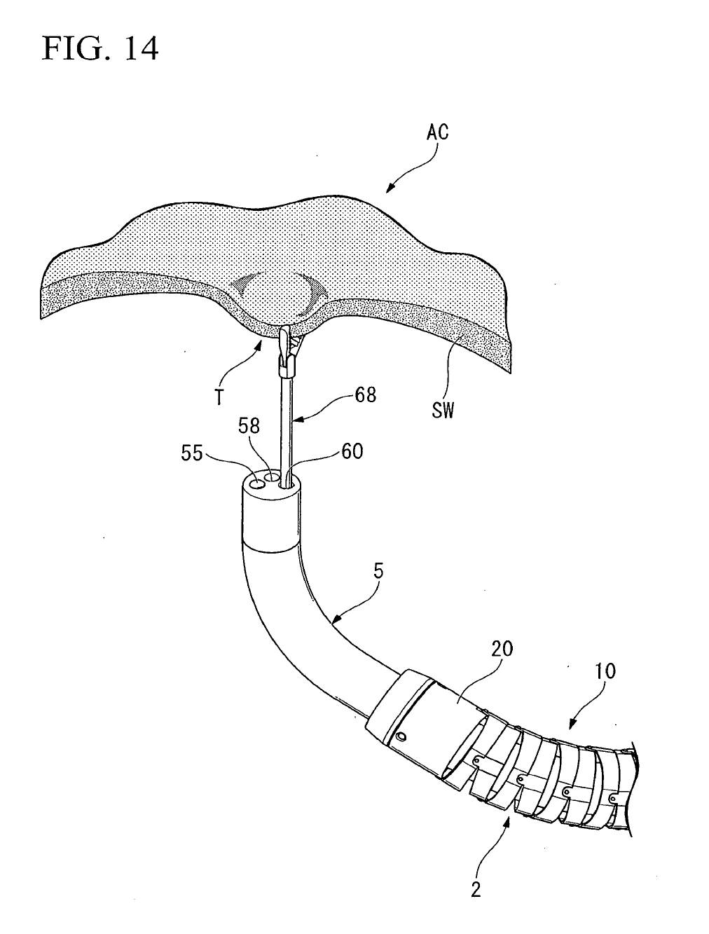

First, in a grasping step (S41), as shown in FIG. 14, the endoscope inserting part 5 is protruded from the distal end part 20 of the overtube 2, and grasping forceps 68 inserted in the treatment instrument insertion channel 60 are further protruded near the incision target site T to grasp the stomach wall SW including the incision target site T. Then, by pulling the grasping forceps 68 into the treatment instrument insertion channel 60, a sufficient space is thereby secured for the abdominal cavity AC on the outer side of the stomach wall SW by making the stomach wall SW concave.

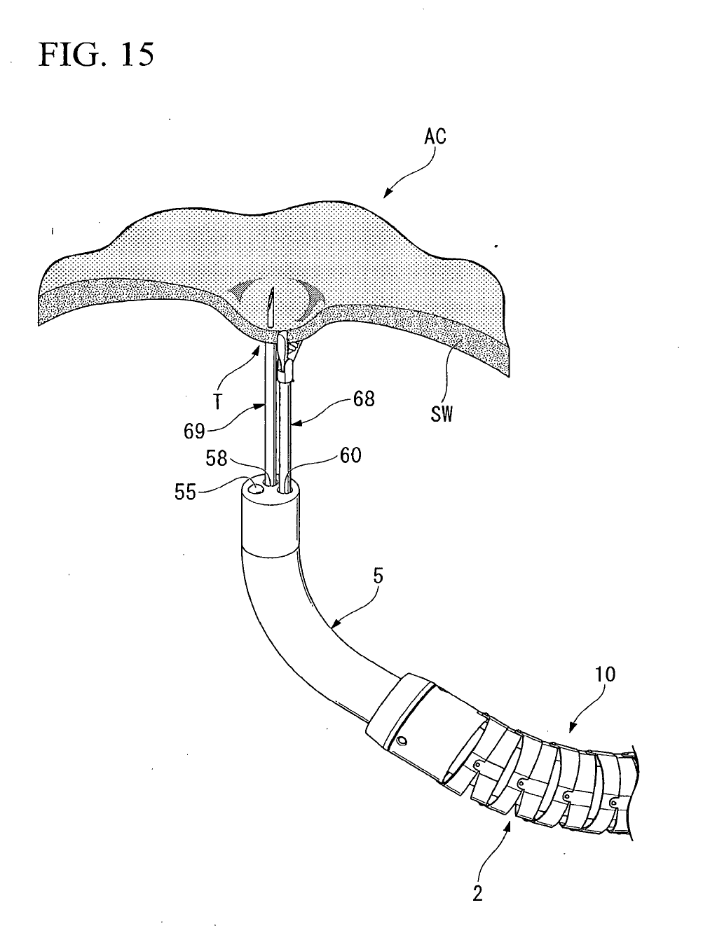

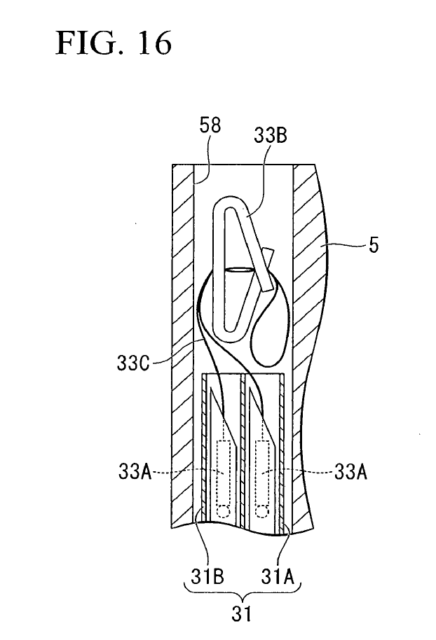

An abdominal cavity insufflating step (S42) is then performed. First, an injection needle 69 connected to the air/water feeding device not shown is inserted through the treatment instrument insertion channel 58 of the endoscope 3. A distal end of the injection needle 69 is then protruded from the distal end and, as shown in FIG. 15, pierced through the stomach wall SW pulled by the grasping forceps 68 and inserted to the abdominal cavity AC. Because the injection needle 69 is pierced with the stomach SW wall being pulled in and a space being secured with the abdominal wall not shown, just the stomach wall SW can be punctured reliably. Air is then fed into the abdominal cavity AC via the injection needle 69 so that the stomach ST and the abdominal wall not shown separate.

The injection needle 69 preferably has a needle length of approximately 12 mm and more preferably has a bendable distal end to enable piercing of the center of the pulled stomach wall. In this case, a bended injection needle has a bending tendency at a distal end and has a bending wire (not shown) that passes from the distal end toward a proximal side in an inward radial direction of the bending tendency. Here, since the treatment instrument insertion channel 58 of the endoscope 3 is disposed at a position of six o'clock to eight o'clock of the endoscope inserting part 5, the incision site is approached from an upward angle in incising the anterior stomach wall SW of the stomach ST that is preferable as the incision site. Accordingly, since the bending tendency faces the center of the bending wire 11 following the bended state of the insertion part 10 of the overtube 2, the center of the stomach wall can be punctured reliably by pulling the bending wire 11 toward the proximal side. In the process of feeding air, the interior of the abdominal cavity AC may be maintained at an appropriate pressure by monitoring and automatic control of the feed air pressure.

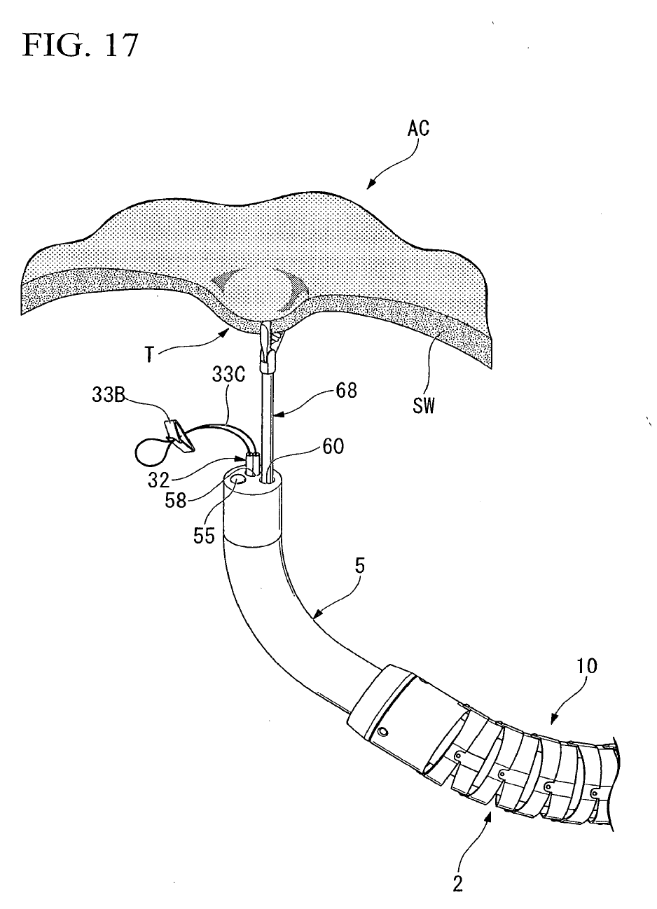

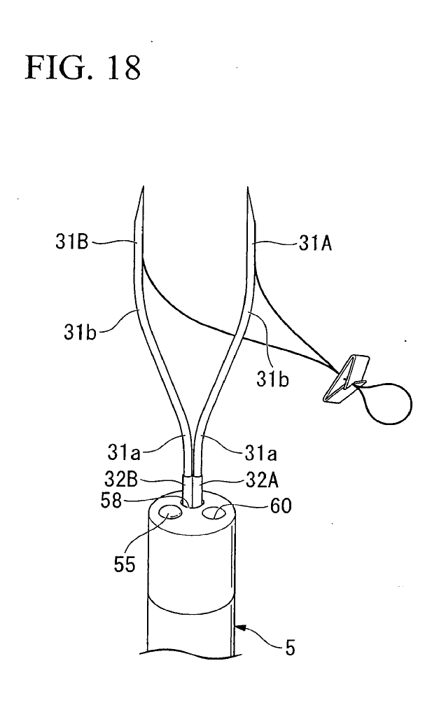

A placing step (S43) is then performed. Here, first the puncture needle 6 is inserted in the treatment instrument insertion channel 58 instead of the injection needle 69. Then, as shown in FIG. 17, in the vicinity of the incision target site T, the distal end of the sheath 32 is protruded from the treatment instrument insertion channel 58 to be disposed near the stomach wall SW. Moreover, the needle manipulating handle 41 is advanced in the direction of the sheath holding part 40 and, as shown in FIG. 18, the first needle part 31A and the second needle part 31B are protruded from the distal end of the sheath 32, extended separated by a predetermined distance, and proceed to pierce the stomach wall SW. At this time, since a space with the stomach wall SW is secured by insufflation of the abdominal cavity AC, it is possible to puncture only the stomach wall SW.

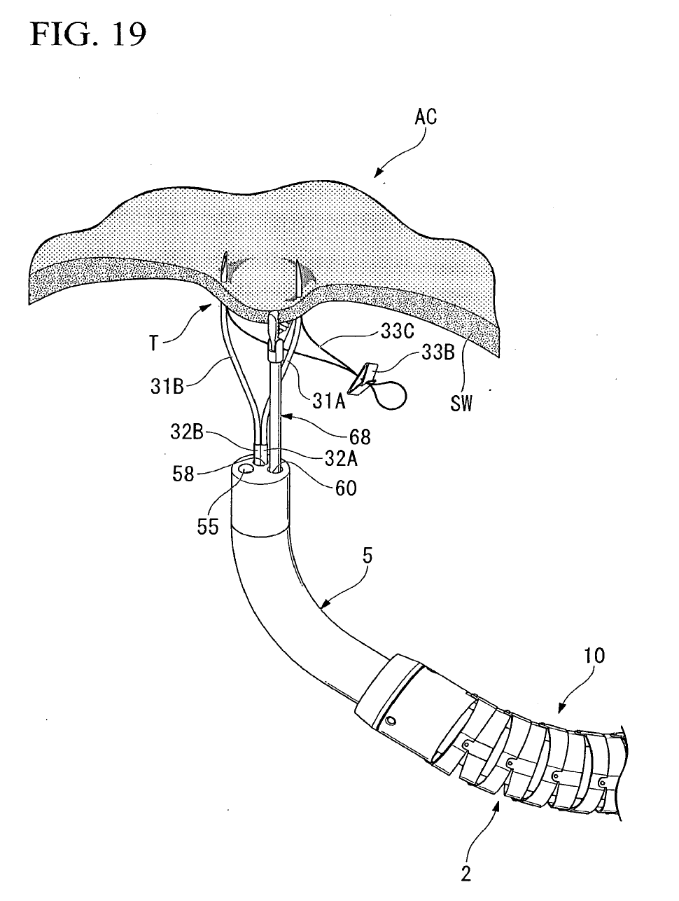

By thus advancing the needle manipulating handle 41, as shown in FIG. 19, two different locations of the stomach wall SW are simultaneously pierced.

The pusher connection part 43 is advanced from this state with respect to the needle manipulating handle 41, and the pusher 35 moves in the distal end direction of the first needle part 31A and the second needle part 31B. At this time, the anchors 33A of the double T-bars 33 are pushed by the pushers 35 to be sent out from within the first needle part 31A and the second needle part 31B to the abdominal cavity AC.

After the anchors 33A of the double T-bars 33 are released, the pusher connection part 43 retracts with respect to the needle manipulating handle 41, and moreover, the needle manipulating handle 41 retracts with respect to the sheath holding part 40, and the first needle part 31A and the second needle part 31B reenter the sheath 32. At this time, the two anchors 33A of the double T-bars 33 open in a T shape due to the bending disposition of the sutures 33C. Thereafter, the entire puncture needle 6 is pulled back to the proximal side, to be withdrawn from the treatment instrument insertion channel 58.

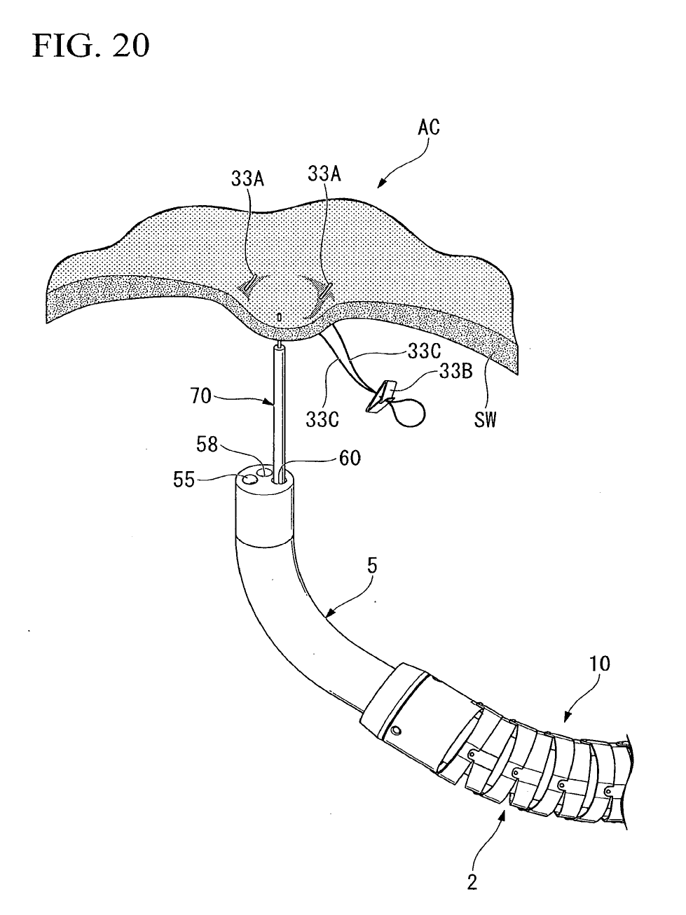

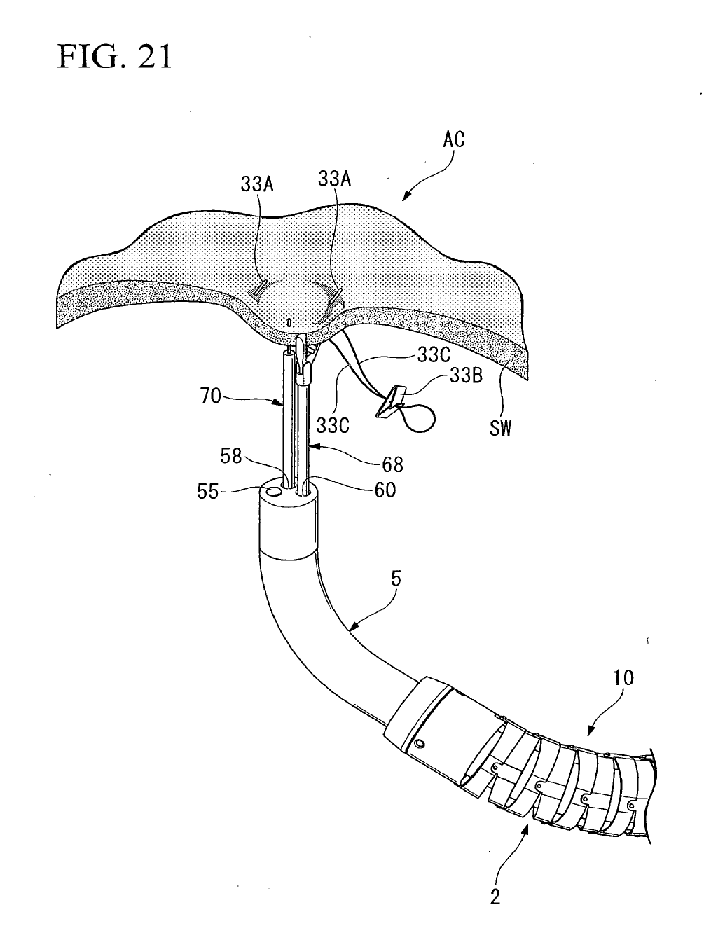

The process then proceeds to an incising step (S50). First, a high-frequency knife 70 is inserted through the treatment instrument insertion channel 60 instead of the grasping forceps 68. At this time, it is confirmed that the connection terminal of the power cord is connected to the connection terminal of the electrode manipulating part not shown. Then, high-frequency power is supplied from a high-frequency power source not illustrated in the state of the distal end of the high-frequency knife 70 abutting the stomach wall SW as shown in FIG. 20. As shown in FIG. 21, the high-frequency knife 70 is inserted through the treatment instrument insertion channel 58 in the state of the grasping forceps 68 inserted through the treatment instrument insertion channel 60. While pulling on the stomach wall SW with the grasping forceps 68, the distal end of the high-frequency knife 70 may be made to abut the stomach wall SW with the placement position of the double T-bars 33 and the incision position in an optimal state.

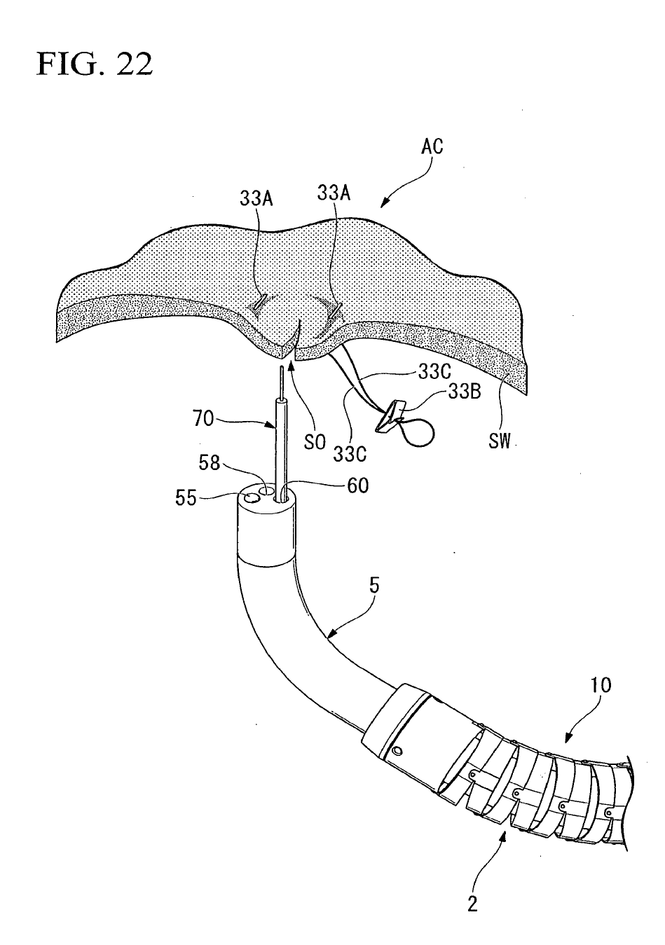

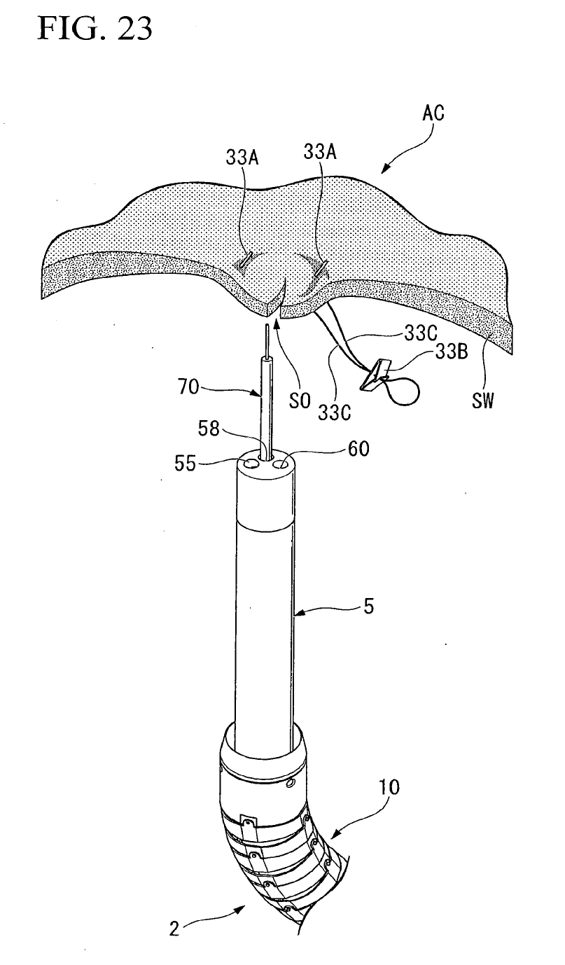

At this time, as shown in FIG. 22 and FIG. 23, the stomach wall SW is incised by the high-frequency knife 70, and an opening SO is formed in the stomach wall SW.

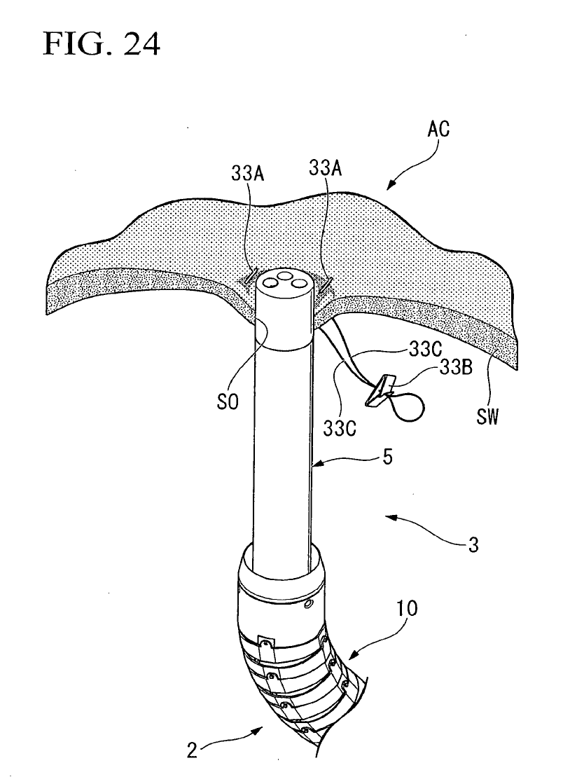

Next, the process proceeds to an introducing step (S60). That is, as shown in FIG. 24, after removing the high-frequency knife 70, the endoscope inserting part 5 of the endoscope 3, which is also an operative device, is introduced into the abdominal cavity AC through the opening SO. If, in this process, relative movement of the insertion part 10 and the endoscope inserting part 5 must be restricted, the endoscope lock button 47 is pressed and contacted against the endoscope inserting part 5 to fix the movement of the endoscope inserting part 5 by the frictional force. Since the endoscope lock button 47 is provided, the endoscope lock button 47 can be manipulated to restrain relative movement of the endoscope 3 with respect to the overtube 2, and the overtube 2 and the endoscope inserting part 5 can thus be inserted into the body simultaneously. Also, since the task of inserting the endoscope 3 can be performed while holding the proximal handle 44 of the overtube 2, an operation in which the insertion part 10 of the overtube 2 is supported by one hand of the operator and the proximal handle 44 is held by the other hand, is enabled, and the operability is thus more improved.

After positioning, a treating step (S70) of performing observation, incision, cell sampling, suturing, or any of other various treatments (medical procedures) is carried out. After performing the treatment, the overtube 2 and the endoscope 3 are removed from the opening SO of the stomach wall SW.

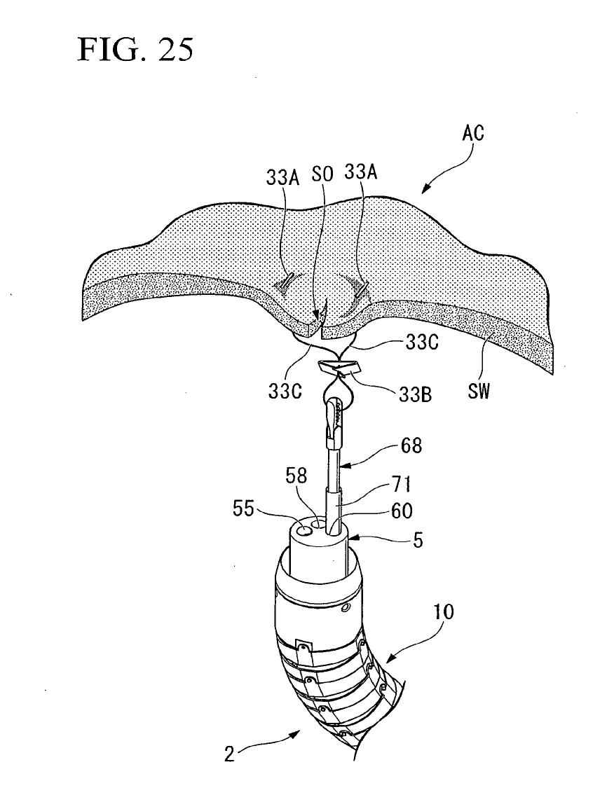

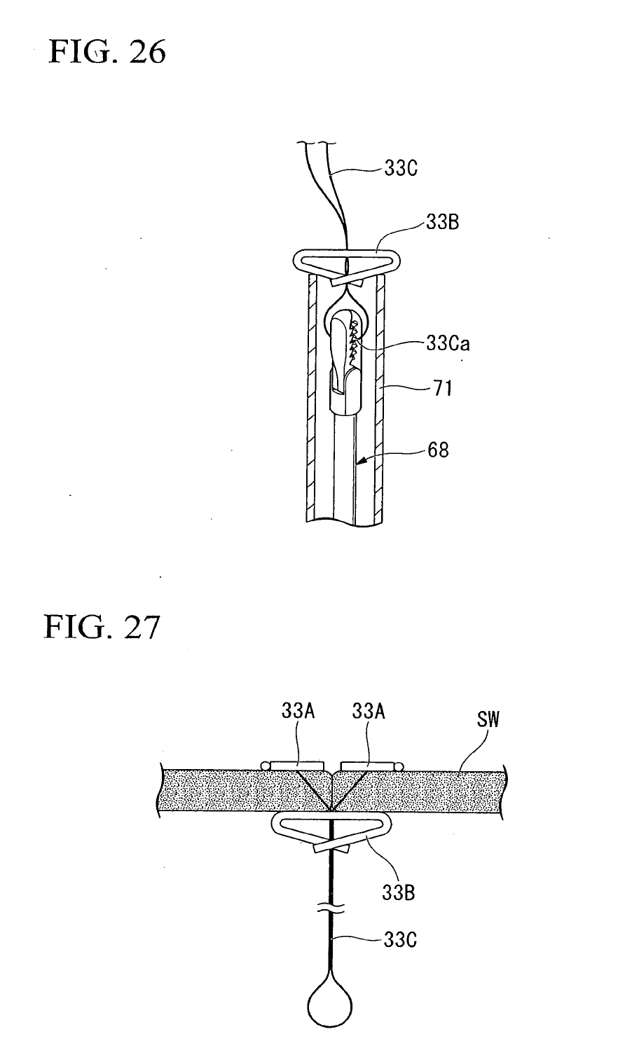

In a suturing step (S80), when removing the endoscope 3 from the opening SO, as shown in FIG. 25, the grasping forceps 68 inserted to freely advance and retract in the outer sheath 32, are protruded with the outer sheath 32 from the treatment instrument insertion channel 60. Then, as shown in FIG. 26, the large diameter part 33Ca of the sutures 33C is held and pulled by the grasping forceps 68 while making the distal end of the outer sheath 32 abut the stopper 33B of the double T-bars 33, which had been placed in advance. Thus, as shown in FIG. 27, by moving the stopper 33B to clinch the stomach wall SW, the opening SO is thereby sutured. Additional double T-bars 33, etc., are provided to perform further suturing if necessary. In this process, since the insufflation is performed in the process of placing the double T-bars 33 at the stomach wall SW, suturing by means of additional double T-bars 33 can be performed readily.

After suturing, the endoscope 3 and the overtube 2 are drawn out of the patient, the pressure applied to the abdominal cavity AC is released, and the surgical procedure is ended.

According to this overtube 2, since the gaps between the joint rings 13 are filled by the inner braid 16, when inserting the endoscope inserting part 5 in the lumen 7, the inner surface of the inner braid 16 serves as a guide so that the endoscope inserting part 5 can be advanced without becoming caught between the joint rings 13. When doing so, since the inner braid 16 is formed by braiding the thin metallic wire 30, deformation from both compression and pulling is possible. Also, when the bending tube 15 bends by the turning of the plurality of joint rings about the connecting shafts 12, the inner braid 16 suitably follows suit, so that it is possible to smoothly bend the bending tube 15.

When curving the bending part 8, by pulling the bending wire 11 toward the proximal side, the joint rings 13 turn at a predetermined angle about the connecting shafts 12 in the sequence in which the joint rings 13 are disposed from the distal end side. Thereby, it is possible to form the bending part 8 having a prescribed curve. On the other hand, to extend the bending part 8 to be straight, the bending wire 11 is loosened. At this time, due to the resiliency of the endoscope inserting part 5, the bent state is straightened. In accordance with this, the bending part 8 can also be straightened. Following this, incising of tissue can be more readily performed.

### Second Embodiment

A second embodiment according to this invention shall now be described with reference to the drawings.

A point of difference of the second embodiment with respect to the first embodiment is that when a bending tube 73 of an overtube 72 according to this embodiment extends in a straight line manner, at least a portion of the peripheral edges of adjacent joint rings 75 overlap in the axial direction so as not to alter the inner diameter of the bending tube 73.

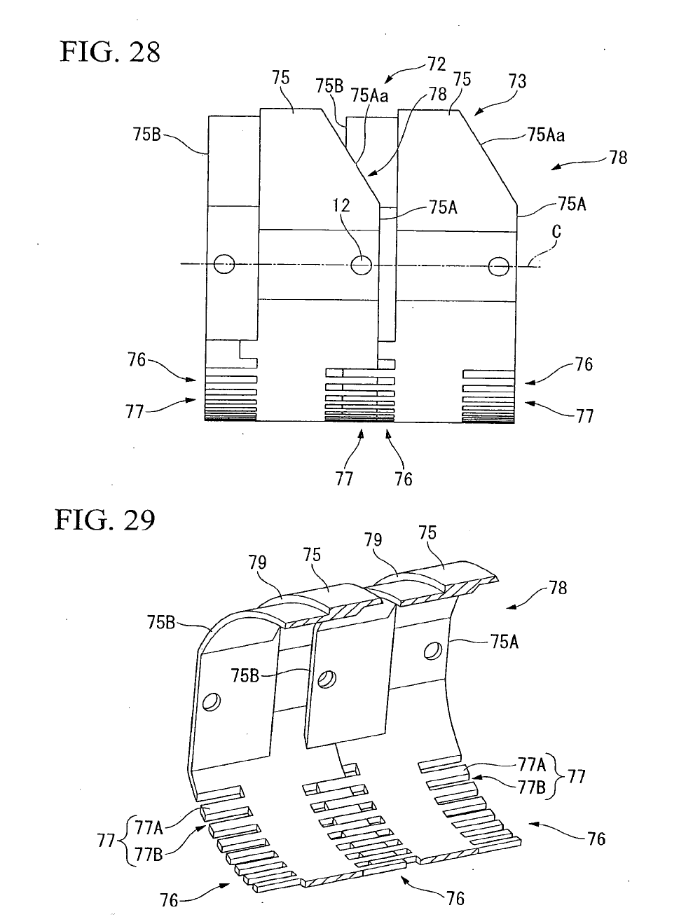

As shown in FIG. 28 and FIG. 29, comb teeth 77 are provided at a specified interval in the circumferential direction in a partial region 76 of the peripheral portion of a proximal end surface 75A and a distal end surface 75B of each joint ring 75.

The comb teeth 77 consist of teeth 77A and slits 77B which are alternately provided so that the comb teeth 77 disposed on the adjacent proximal end surface 75A of the joint ring 75 and the distal end surface 75B of the opposing joint ring 75 mesh. The teeth 77A are of a length so that the meshing of the comb teeth 77 is maintained even when the proximal end surface 75A and a distal end surface 75B come apart by the curvature of the bending tube 15 at a predetermined angle.

In a separate region 78 in which the comb teeth 77 are not provided, a portion 75Aa of the proximal end surface 75A that becomes the inner side in the radial direction during curving is formed slanting with respect to the distal end surface 75B. In this region 78, a step 79 is formed so that the distal end surface 75B has a smaller diameter than the proximal end surface 75A by an amount corresponding to the wall thickness of the joint ring 75. Thereby, when the bending tube 15 bends, the distal end surface 75B of another joint ring 75 that is adjacent to the proximal end surface 75A of the joint ring 75 becomes fitted on the inner side.

Actions of the present embodiment shall now be described in line with a medical procedure performed via a natural orifice using the overtube 2 similarly to the first embodiment.

First, the inserting step (S10) is carried out similarly to the first embodiment.

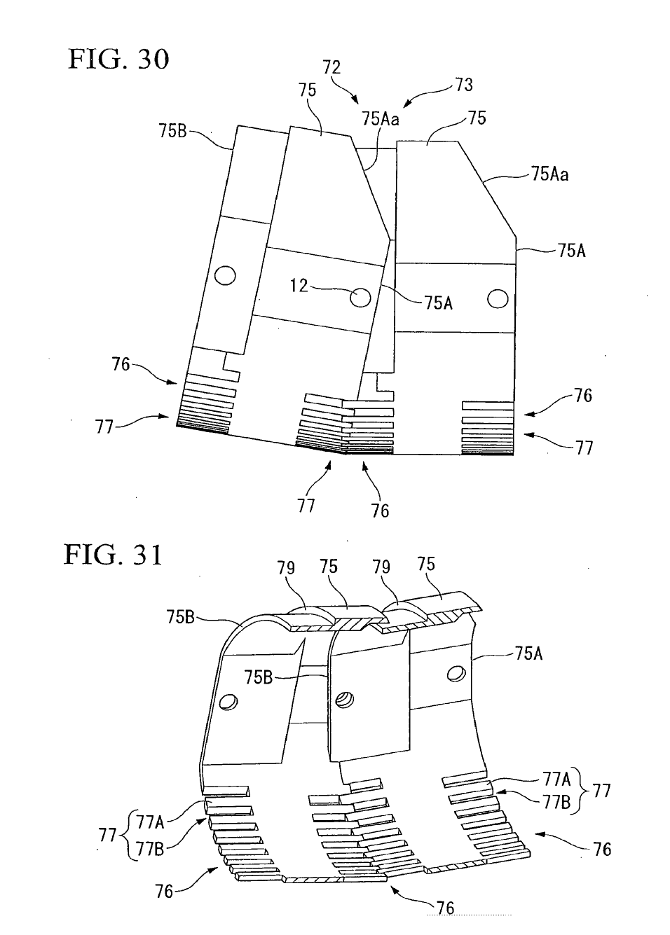

Here, even when the bending tube 15 is bent, as shown in FIG. 30 and FIG. 31, the comb teeth 77 that are disposed on the distal end surface 75B of one joint ring 75 engage with the comb teeth 77 that are disposed on the distal end surface 75B of another joint ring 75 adjacent thereto. For this reason, gaps are not formed between the joint rings 75, and so when inserting the endoscope 3 in the overtube 2, even if the distal end thereof passes through the bending tube 73, the distal end of the endoscope inserting part 5 does not enter a gap between the joint rings 75. Accordingly, the endoscope inserting part 5 moves smoothly in the lumen 7.

Afterward, the steps from the distending step (S20) to the suturing step (S80) are performed similarly to the first embodiment. After the surturing, the endoscope 3 is removed from the patient, the pressure applied to the abdominal cavity AC is released, and the surgical procedure is ended.

According to this overtube 2, since there are no gaps between the joint rings 75 regardless of whether there is bending or not, the endoscope inserting part 5 can be smoothly inserted into the insertion part 10 similarly to the first embodiment. Also, since there is no inner braid 16 such as that of the overtube 2 according to the first embodiment, it is possible to secure a lumen with a greater diameter than the diameter of the lumen according to the first embodiment.

The scope of the art of this invention is not restricted to the embodiments described above, and various changes can be added within a range that does not fall outside the spirit of this invention.

For example, through in the above embodiment, a flexible endoscope is used as an observation device, this invention is not limited thereto and, for example, a so-called capsule endoscope may be placed inside the body, and while observing the interior of the body using the endoscope, an insertion part of a treatment device that does not have an observation device may be inserted through the overtube to perform the desired surgical procedure.

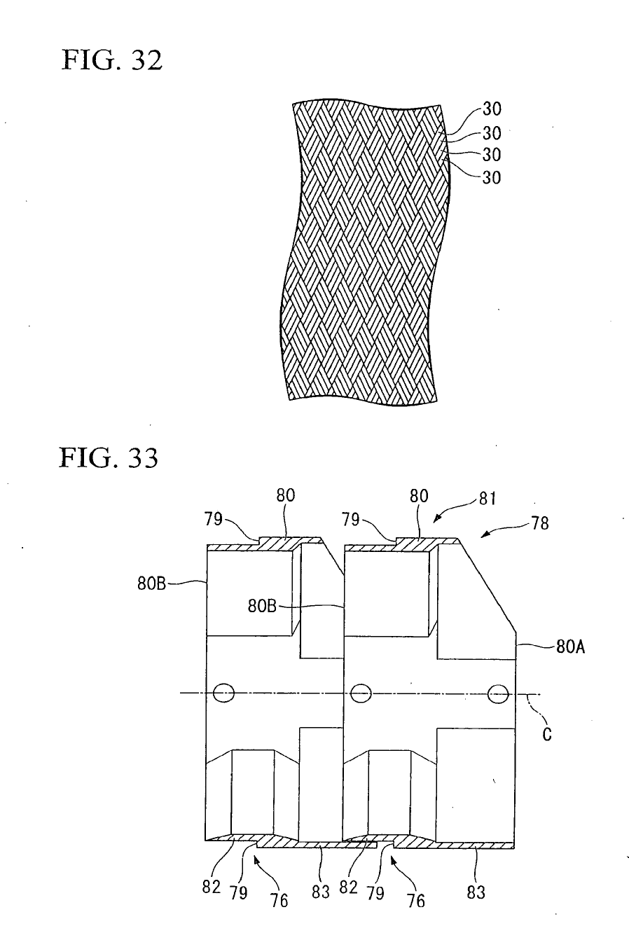

Also, in the first embodiment, the inner braid 16 and the outer braid 17 are formed by braiding one thin metallic wire 30 so as to intersect with the central axis C of the lumen 7, but are not limited thereto. For example, as shown in FIG. 32, a plurality of the thin metallic wires 30 may be braided in a similar direction. In this case, although the movement angle is further constrained than in the case of a single wire, the strength is increased, and the required rigidity can be ensured. Also, by filling resin between the thin metallic wires 30, airtightness and watertightness may be ensured. Also, the thin wires may be a nonmetal instead of metal. Also, the surface of the thin metallic wires 30 or the entire inner braid may be coated with a resin or ceramics.

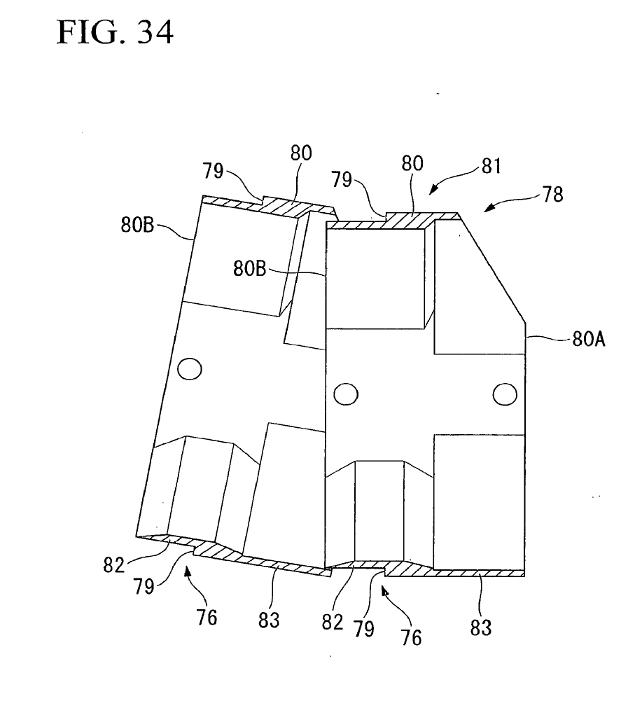

Also, in the second embodiment, as shown in FIG. 33, a bending tube 81 may be constituted by a part of adjacent joint rings 80 overlapping in the radial direction of the joint rings 80. In this case, in the partial region 76 of the joint ring 75, the step 79 similar to the separate region 78 may be provided instead of the comb teeth 77 provided in the partial region 76 of the joint ring 75. That is, the joint ring 80 is provided with a small diameter part 82 of the distal end side and a large diameter part 83 on the proximal end side that fits with the small diameter part 82 of the adjacent joint ring 80. Here, as shown in FIG. 34, the length of the small diameter part 82 and the large diameter part 83 along the central axis C is a length that is capable of maintaining the mutual fitting so that gaps are not formed between the joint rings 80 even when the bending tube bends at a predetermined curvature. Accordingly, when inserting the endoscope 3 in the overtube 2, even if the distal end thereof passes through the bending tube 81, the distal end of the endoscope inserting part 5 does not enter a gap between the joint rings 80.

What is claimed is:

1. An overtube comprising:
an insertion part that is inserted into a subject and has a lumen through which a device inserting part of a device whose distal end bends freely for performing a medical procedure inside a body is removably inserted and a bending part that bends the distal end side of the lumen,
wherein the bending part is provided with a bending tube having a plurality of joint rings that are connected via connecting shafts along the lumen to freely turn and a braided tube that is disposed on the inner side of the bending tube and forms the periphery of the lumen.

2. An overtube comprising:
an insertion part that is inserted into a subject and has a lumen through which a device inserting part of a device whose distal end bends freely for performing a medical procedure inside a body is removably inserted and a bending part that includes a bending tube having a plurality of joint rings that are connected via connecting shafts along the lumen to freely turn,
wherein at least a portion of the periphery of adjacent joint rings that constitute the bending tube overlap so as not to change the inner diameter of the bending tube.

3. An overtube comprising:
an insertion part that has a lumen through which a device inserting part of a device whose distal end bends freely for performing a medical procedure inside a body is removably inserted, a bending part that includes a bending tube having a plurality of joint rings that are connected via connecting shafts along the lumen to freely turn, and a sealing means blocking gaps between adjacent joint rings.

4. An endoscopic treatment system comprising:
an overtube according to any one of claims 1 to 3;
a device inserting part that is inserted in the insertion part of the overtube in which is provided a treatment instrument insertion channel whose distal end is opened; and
a puncture needle that is inserted in the treatment instrument insertion channel, with the distal end thereof splitting apart to be wider than the inner diameter of the treatment instrument insertion channel.

5. The overtube according to claim 2, wherein
at least a portion of the adjacent joint rings overlap in the axial direction of the bending tube.

6. The overtube according to claim 2, wherein
at least a portion of the adjacent joint rings overlap in the radial direction of the joint rings.

7. The overtube according to any one of claims 1 to 3, further comprising:
a tube-shaped distal end part that is connected to the distal end of the bending part; and
a bending wire whose distal end is connected to the distal end part and is provided to pass through the joint rings;
wherein only one bending wire is disposed.
